(19) United States
(12) Patent Application Publication
(10) Pub. No.: US 2015/0045623 A1
(43) Pub. Date: Feb. 12, 2015

(54) SURGICAL SEAL ASSEMBLY INCLUDING A FLOATING SEAL STRUCTURE

(71) Applicant: Covidien LP, Mansfield, MA (US)

(72) Inventor: Gregory Fischvogt, Hamden, CT (US)

(21) Appl. No.: 14/309,095

(22) Filed: Jun. 19, 2014

Related U.S. Application Data
(60) Provisional application No. 61/863,943, filed on Aug. 9, 2013.

(51) Int. Cl. A61B 17/02

(52) U.S. Cl. CPC A61B 17/02
USPC 600/201

(57) ABSTRACT

A surgical access device includes a seal assembly having an upper housing member, a lower housing member, and a receiving member. The receiving member defines a central longitudinal axis and has a longitudinal passage for receiving a surgical object therethrough. The surgical access device also includes a first seal secured between the upper housing member and the lower housing member and a second seal positioned between the upper housing member and the receiving member. The first seal provides a biasing force to maintain the second seal in contact with the upper housing member to provide a sealing relationship therewith.

### CROSS-REFERENCE TO RELATED APPLICATIONS

This application claims the benefit of and priority to U.S. Provisional Patent Application No. 61/863,943, filed Aug. 9, 2013, the entire disclosure of which is incorporated by reference herein.

### BACKGROUND

1. Technical Field

The present disclosure relates to a surgical access device and, more particularly, but not exclusively, relates to a surgical access device having a floating seal structure.

2. Background of Related Art

Minimally invasive surgical procedures including both endoscopic and laparoscopic procedures permit surgery to be performed on organs, tissues and vessels far removed from an opening through the tissue. Laparoscopic and endoscopic procedures generally require that any instrumentation inserted into the body be sealed, i.e., provisions must be made to ensure that gases do not enter or exit the body through the incision as, for example, in surgical procedures in which the surgical region is insufflated. These procedures typically employ surgical instruments which are introduced into the body through a cannula. The cannula has a housing at a proximal end thereof in which a seal assembly is mounted. The seal assembly provides a substantially fluid tight seal about the instrument to preserve the integrity of the established pneumoperitoneum.

Minimally invasive procedures have several advantages over traditional open surgery, including less patient trauma, reduced recovery time, reduced potential for infection, etc. However, there remains room for improvement in apparatus used to guide a surgical instrument through a seal assembly in a more efficient and efficacious manner.

### SUMMARY

The following presents a simplified summary of the claimed subject matter in order to provide a basic understanding of some aspects of the claimed subject matter. This summary is not an extensive overview of the claimed subject matter. It is intended to neither identify key or critical elements of the claimed subject matter nor delineate the scope of the claimed subject matter. Its sole purpose is to present some concepts of the claimed subject matter in a simplified form as a prelude to the more detailed description that is presented later.

According to one aspect of the present disclosure, a surgical access device is provided. The surgical access device includes a seal assembly having an upper housing member, a lower housing member, and a receiving member defining a central longitudinal axis and having a longitudinal passage for receiving at least one surgical object therethrough. The surgical access device also includes a first seal disposed within the seal assembly, the first seal secured between the upper housing member and the lower housing member and a second seal disposed within the seal assembly, the second seal movably secured between the upper housing member and the receiving member. The first seal provides a biasing force to maintain the second seal in contact with the upper housing member to create a sealing relationship therewith.

In one exemplary embodiment, the first seal is a flat seal and the second seal is an O-ring.

In another exemplary embodiment, the upper housing member, the lower housing member, and the first seal move in unison, as a single unitary component, and float in a planar motion upon application of a lateral force.

In yet another exemplary embodiment, the receiving member and the second seal move independently of the upper housing member, the lower housing member, and the first seal upon application of a lateral force.

In another exemplary embodiment, an upper portion of the receiving member defines an angular opening therethrough to facilitate angular reception of the at least one surgical object.

In one exemplary embodiment, an upper portion of the receiving member is configured to be attached to a proximal end of the seal assembly.

In yet another exemplary embodiment, a distal end of the seal assembly includes a duckbill seal. A space between the first seal and the duckbill seal is devoid of any intermediate components.

In yet another exemplary embodiment, movement of the second seal is restricted within a region defined by an upper portion of the upper housing member and a ridge of the receiving member.

According to another aspect of the present disclosure, a surgical access device is provided. The surgical access device includes a seal assembly having a housing with an upper housing member and a lower housing member, the seal assembly defining a central longitudinal axis and having a longitudinal passage for receiving at least one surgical object therethrough. The surgical access assembly also includes a first seal disposed within the seal assembly, the first seal positioned between the upper housing member and the lower housing member and a second seal disposed within the seal assembly, the second seal positioned between the upper housing member and the spherical member. The first seal provides a biasing force to maintain the spherical member in contact with second seal to create a sealing relationship therebetween.

According to another aspect of the present disclosure, an access assembly is provided. The access assembly includes a housing formed of an upper retaining portion and a lower retaining portion, a first seal secured at the lower retaining portion of the housing, and a second seal secured at the upper retaining portion of the housing. The first seal imparts a biasing force to create a sealing relationship between the upper retaining portion and the second seal.

In another aspect of the present disclosure, a method for performing a surgical procedure is provided. The method includes the step of providing a seal assembly having an upper housing member, a lower housing member, and a receiving member configured to form a housing, the receiving member defining a central longitudinal axis and having a longitudinal passage for receiving at least one surgical object therethrough. The method also includes the steps of providing a first seal disposed within the seal assembly, the first seal secured between the upper housing member and the lower housing member and providing a second seal disposed within the seal assembly, the second seal movably secured between the upper housing member and the receiving member. The first seal provides a biasing force to move the second seal toward the upper housing member to create a sealing relationship therewith.

In another aspect of the present disclosure, a method for performing a surgical procedure is provided. The method includes the steps of providing a housing formed of an upper retaining portion and a lower retaining portion, providing a first seal secured at the lower retaining portion of the housing, and providing a second seal secured at the upper retaining portion of the housing. Upon application of a force, the first seal imparts a biasing force to displace the second seal to create a sealing relationship between the upper retaining portion and the second seal.

Further scope of applicability of the present disclosure will become apparent from the detailed description given hereinafter. However, it should be understood that the detailed description and specific examples, while indicating embodiments of the present disclosure, are given by way of illustration only, since various changes and modifications within the spirit and scope of the present disclosure will become apparent to those skilled in the art from this detailed description.

### BRIEF DESCRIPTION OF THE DRAWINGS

The accompanying drawings, which are incorporated in and constitute a part of this specification, illustrate embodiments of the disclosure and, together with a general description of the disclosure given above, and the detailed description of the embodiment(s) given below, serve to explain the principles of the disclosure, wherein:

FIGS. 1 and 2 are perspective views of a surgical access device, in accordance with the present disclosure;

FIG. 3 is a side, cross-sectional view of the surgical access device of FIG. 1 illustrating the seal assembly, in accordance with the present disclosure;

FIG. 4 is a side, cross-sectional view illustrating a seal assembly having the first seal and the second seal, in accordance with the present disclosure;

FIG. 5 is a side, cross-sectional view of the seal assembly incorporating the first seal and the second seal of FIG. 4, in accordance with an embodiment of the present disclosure;

FIGS. 6 and 7 are side cross-sectional views of the seal assembly of FIG. 5 connected to a cannula assembly, where a surgical instrument is inserted therethrough to bias the seal assembly to the left and the right, in accordance with the present disclosure;

FIG. 8 is a perspective view of a spherical member positioned within a housing, in accordance with a second embodiment of the present disclosure;

FIG. 9 is a side cross-sectional view of a seal assembly incorporating the spherical member of FIG. 8, in accordance with the present disclosure; and

FIGS. 10 and 11 are side cross-sectional views of the seal assembly of FIG. 9 connected to a cannula assembly, where a surgical instrument is inserted therethrough to bias the spherical member to the left and the right, in accordance with the present disclosure.

The figures depict embodiments of the present disclosure for purposes of illustration only. One skilled in the art will readily recognize from the following discussion that alternative embodiments of the structures and methods illustrated herein may be employed without departing from the principles of the present disclosure described herein.

### DETAILED DESCRIPTION

Particular embodiments of the present disclosure are described hereinbelow with reference to the accompanying drawings. However, it is to be understood that the disclosed embodiments are merely exemplary of the disclosure and may be embodied in various forms. Well-known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure.

For the purposes of promoting an understanding of the principles of the present disclosure, reference will now be made to the exemplary embodiments illustrated in the drawings, and specific language will be used to describe the same. It will nevertheless be understood that no limitation of the scope of the present disclosure is thereby intended. Any alterations and further modifications of the inventive features illustrated herein, and any additional applications of the principles of the present disclosure as illustrated herein, which would occur to one skilled in the relevant art and having possession of this disclosure, are to be considered within the scope of the present disclosure.

The cannula assembly of the present disclosure, in combination with a seal system internal to the cannula assembly, provides a substantial seal between a body cavity of a patient and the outside atmosphere before, during and after insertion of a surgical instrument through the cannula assembly. Moreover, the seal assembly of the present disclosure is capable of accommodating surgical instruments of varying diameters, e.g., from 5 mm to 15 mm, by providing a fluid tight seal with each instrument inserted through the seal assembly. The flexibility of the present seal assembly greatly facilitates endoscopic surgery where a variety of instruments having differing diameters may be used during a single surgical procedure.

The seal assembly receives various types of instrumentation adapted for insertion through a trocar and/or cannula assembly while maintaining a fluid tight interface about the instrumentation to preserve the atmospheric integrity of a surgical procedure from gas and/or fluid leakage. Specifically, the seal assembly accommodates angular manipulation of the surgical instrument relative to the seal housing axis. This feature of the present disclosure desirably minimizes the entry and exit of gases and/or fluids to/from the body cavity. Examples of instrumentation include clip appliers, graspers, dissectors, retractors, staplers, laser probes, photographic devices, endoscopes and laparoscopes, tubes, and the like. Such instruments will be collectively referred to herein as “instruments or instrumentation.”

The word “exemplary” is used herein to mean “serving as an example, instance, or illustration.” Any embodiment described herein as “exemplary” is not necessarily to be construed as preferred or advantageous over other embodiments. The word “example” may be used interchangeably with the term “exemplary.”

Embodiments of the presently disclosed apparatus will now be described in detail with reference to the drawings, in which like reference numerals designate identical or corresponding elements in each of the several views. As used herein, the term “distal” refers to that portion of the tool, or component thereof which is farther from the user while the term “proximal” refers to that portion of the tool or component thereof which is closer to the user.

Reference will now be made in detail to embodiments of the present disclosure. While certain embodiments of the present disclosure will be described, it will be understood that it is not intended to limit the embodiments of the present disclosure to those described embodiments. To the contrary, reference to embodiments of the present disclosure is intended to cover alternatives, modifications, and equivalents as may be included within the spirit and scope of the embodiments of the present disclosure as defined by the appended claims.

For exemplary purposes, the access apparatus will be described in terms of a cannula assembly, which is adapted for introduction, typically utilizing a trocar, within the abdominal cavity during a laparoscopic surgical procedure. However, it is appreciated that the access apparatus may be any apparatus suitable for introduction and passage of surgical objects into underlying tissue including, e.g., catheters, trocar assemblies, endoscopic portals, hand access devices, etc., through an incision or through a natural body opening.

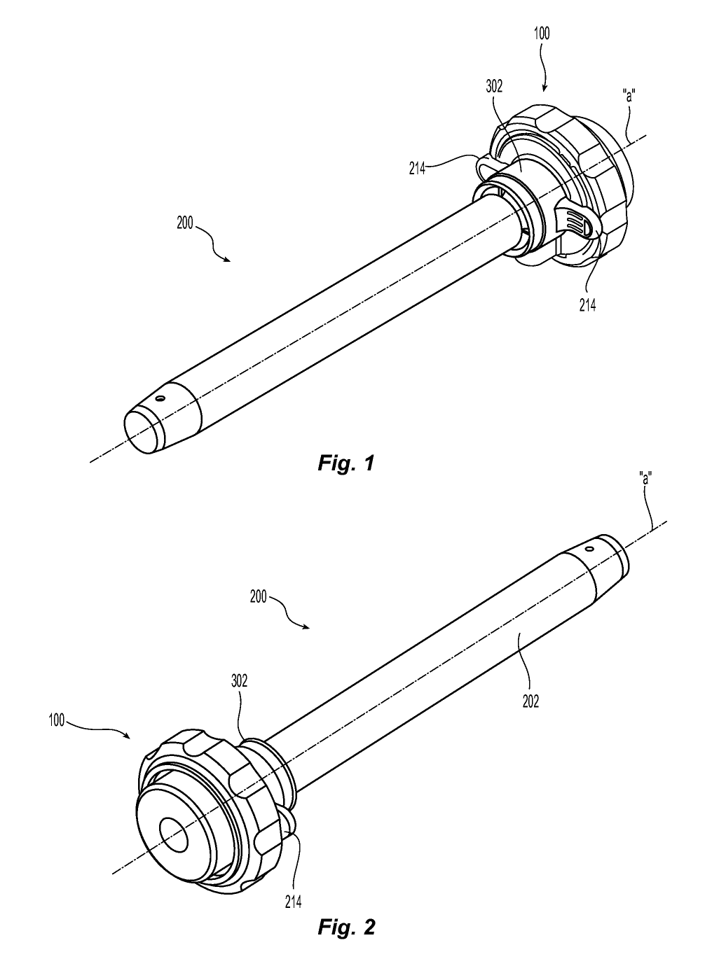

Referring now to the drawings, in which like reference numerals identify identical or substantially similar parts throughout the several views, FIGS. 1 and 2 illustrate the seal access device 100 of the present disclosure mounted to cannula assembly 200. Cannula assembly 200 may be any conventional cannula suitable for the intended purpose of accessing a body cavity and permitting introduction of instruments therethrough. Cannula assembly 200 is particularly adapted for use in laparoscopic surgery where the peritoneal cavity is insufflated with a suitable fluid, e.g., CO₂, to raise the cavity wall from the internal organs therein. Cannula assembly 200 is typically used with an obturator assembly (not shown), which is a sharp pointed instrument positionable within the passageway of the cannula assembly 200. The obturator assembly is utilized to penetrate the abdominal wall and then subsequently be removed from the cannula assembly 200 to permit introduction of the surgical instrumentation utilized to perform the procedure. Seal assembly of the surgical access device 100 further includes diametrically opposed housing grips 214 dimensioned and arranged for gripping engagement by the fingers of the user.

Cannula assembly 200 includes cannula sleeve 202 and cannula housing 302 mounted to an end of the sleeve 202. Cannula sleeve 202 defines a longitudinal axis “a” extending along the length of sleeve 202. Sleeve 202 further defines an internal longitudinal passage dimensioned to permit passage of surgical instrumentation. Sleeve 202 may be formed of stainless steel or other rigid materials, such as a polymeric material or the like. Sleeve 202 may be clear or opaque. The diameter of sleeve 202 may vary, but typically ranges from 10 to 15 mm for use with the seal assembly 100 of the present disclosure.

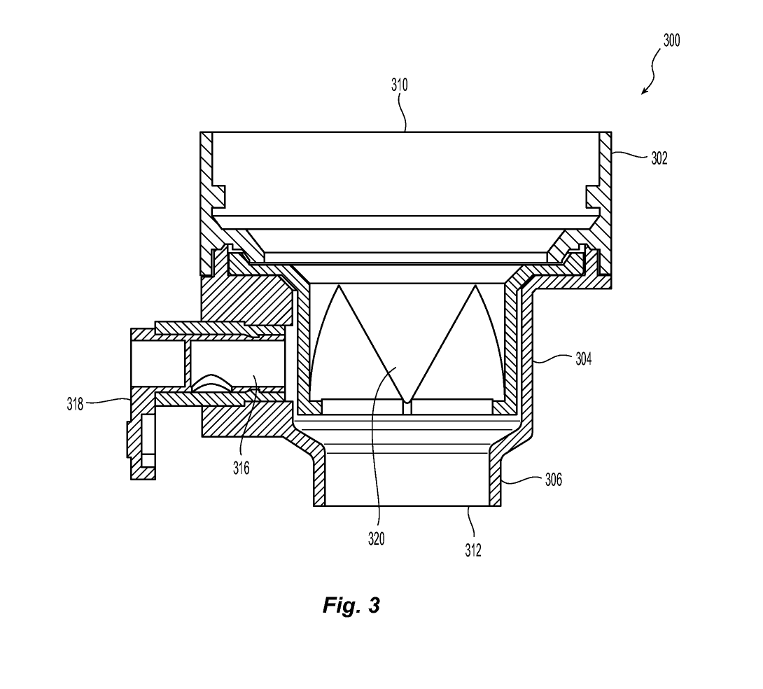

Referring to FIG. 3, a side cross-sectional view of a portion of a cannula assembly 200 of FIG. 2, in accordance with an embodiment of the present disclosure is presented.

The cannula assembly 200 includes a housing 300 having an upper cannula housing 302 and a lower cannula housing 304 connected thereto. The lower cannula housing 304 is connected to a cannula sleeve 306 that extends distally from the lower cannula housing 304. The housing 300 includes an opening 310 at the upper cannula housing 302 for receiving surgical instrumentation therethrough. The cannula sleeve 306 includes an opening 312 at a distal end thereof where the surgical instrumentation exits into, for example, a body cavity of a patient. The lower cannula housing 304 may include a duck bill seal 320 therein, which tapers distally and inwardly to a sealed configuration. Moreover, lower cannula housing 304 may include a luer fitting 316 positioned within a port opening. Luer fitting 316 is adapted for connection to a supply of insufflation fluid and incorporates valve 318 to selectively open and close the passage of luer fitting 316.

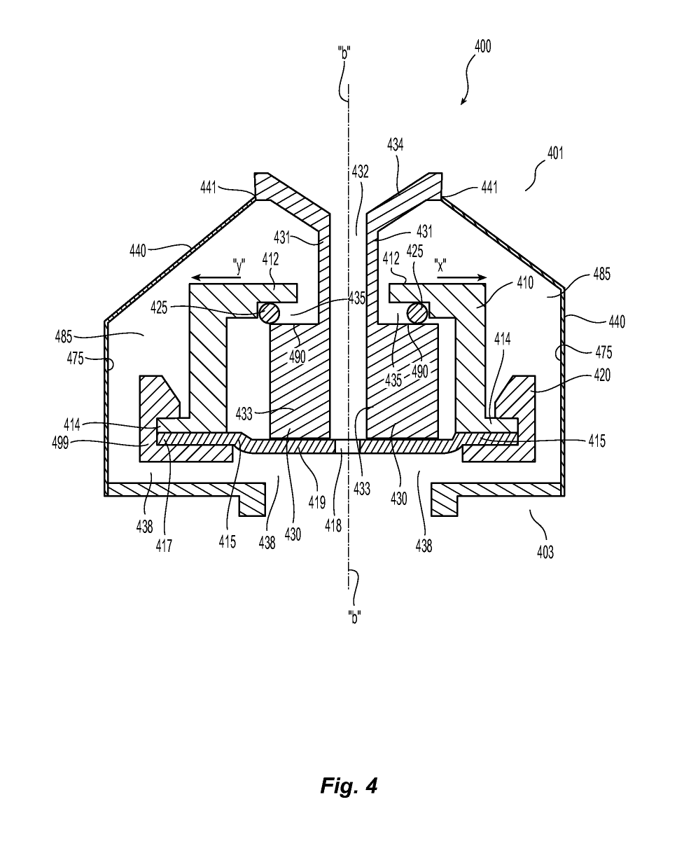

FIG. 4 is a cross-sectional view illustrating a seal assembly 400 having a first seal 415 and a second seal 425, in accordance with the present disclosure.

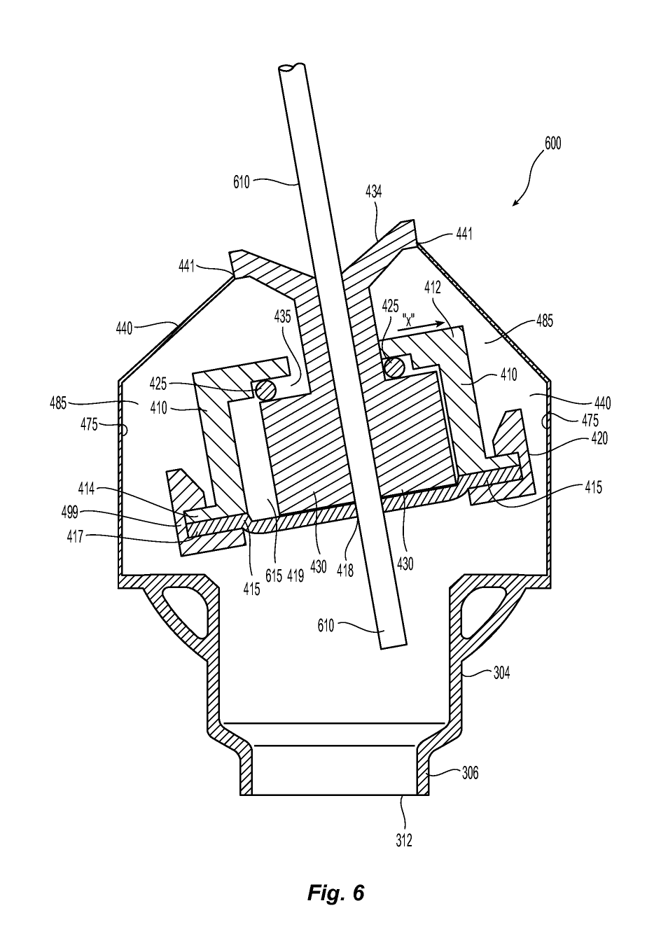

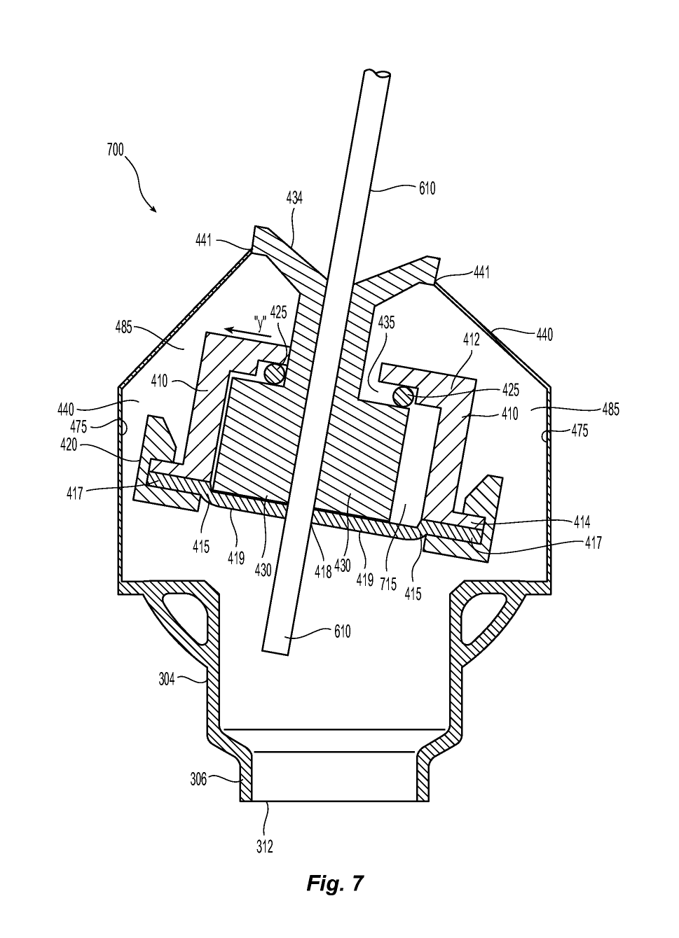

In FIG. 4, a seal assembly 400 has an upper housing member 410, a lower housing member 420, and a receiving member 430 configured to form a housing 440, the receiving member 430 defining a central longitudinal axis “b” and having a longitudinal passage 432 for receiving at least one surgical object (see FIGS. 6 and 7) therethrough.

The first seal 415 is disposed within the seal assembly 400, the first seal 415 secured between the upper housing member 410 and the lower housing member 420. The second seal 425 is disposed within the seal assembly 400, the second seal 425 movably positioned between the upper housing member 410 and the receiving member 430. The first seal 415 provides a biasing force to move the second seal 425 toward the upper housing member 410 to provide a sealing relationship therewith. The second seal 425 rests on a ledge 490 within gap 435.

The first seal 415 is a substantially flat instrument seal, whereas the second seal 425 is an O-ring. In the exemplary embodiments, the upper housing member 410, the lower housing member 420, and the first seal 415 move in unison, as a single unitary component, and float in a planar motion. In contrast, the receiving member 430 and the second seal 425 move independently of the upper housing member 410, the lower housing member 420, and the first seal 415.

The receiving member 430 includes an upper section 431 and a lower section 433. The upper section 431 of the receiving member 430 defines an angular opening 434 therethrough to facilitate angular reception of the at least one surgical object (see FIGS. 6 and 7). The upper section 431 of the receiving member 430 is configured to be attached to a proximal end 401 of the seal assembly 400. A space 438 below the first seal 415 is devoid of any intermediate components. In other words, the upper housing member 410 and the lower housing member 420 appear to float or to be suspended within the seal assembly 400. The receiving member 430 is attached or connected to an upper portion 441 of the housing 440. Thus, the upper housing member 410 and the lower housing member 420 do not directly connect or attach to the housing 440 of the seal assembly 400. Additionally, only upper housing member 410 is directly attached to the receiving member 430. The lower housing member 420 does not directly contact or engage with the receiving member 430.

As noted above, the receiving member 430 includes an upper section 431 and a lower section 433. The upper section 431 is configured to attach to the housing 440 at upper portion 441, whereas the lower section 433 is configured to be positioned directly between the first seal 415 and the second seal 425. In fact, a gap 435 is defined above the lower section 433 to accommodate the second seal 425. The gap 435 may extend the length of the lower section 433. Stated differently, movement of the second seal 425 is restricted within gap 435 defined by an upper portion or section 431 of the upper housing member 410 and a ridge or upper section 431 of the receiving member 430 to prevent escape of insufflation gases. Stated differently, gap 435 is defined between ledge 490 and upper component 412 of upper housing member 410.

The upper housing member 410 may include an upper component 412 and a lower component 414. The upper component 412 and the lower component 414 may be substantially perpendicular to the central longitudinal axis “b” defined by the receiving member 430. In other words, the upper component 412 and the lower component 414 may be horizontal components, such that the upper housing member 410 has a “Z-shaped” configuration. The upper component 412 is configured to engage the second seal 425, as well as the receiving member 430 (i.e., the upper section 431), whereas the lower component 414 is configured to engage the first seal 415, as well as the lower housing member 420. In fact, the lower component 414 is sandwiched between the lower section 433 of the receiving member 430, a portion of the first seal 415, and a portion of the lower housing member 420.

The first seal 415 may include a first end 417, a central portion 419 and an opening 418. The first end 417 is secured between the lower component 414 of the upper housing member 410 and a portion 499 of the lower housing member 420, whereas the central portion 419 engages the lower section 433 of the receiving member 430. The entire bottom portion of the lower section 433 of the receiving member engages the central portion 419 of the first seal 415. It is noted that the opening 418 of the first seal 415 is smaller than the passage 432 in order to create a proper sealing relationship between an instrument 610 (see FIGS. 6 and 7) inserted through the passage 432 and the opening 418.

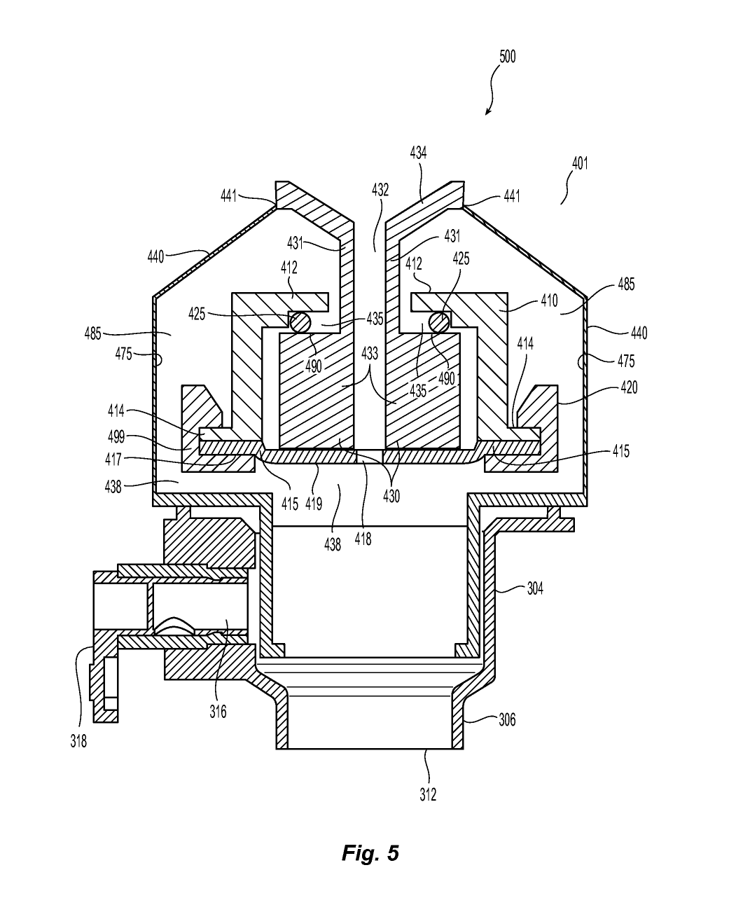

Referring to FIG. 5, a cross-sectional view 500 of the seal assembly 400 incorporating the first seal 415 and the second seal 425 of FIG. 4, in accordance with an embodiment of the present disclosure is presented.

Referring to FIGS. 6 and 7, side cross-sectional views 600, 700 of the seal assembly 500 of FIG. 5 connected to a cannula assembly 100 are presented, where a surgical instrument 610 is inserted therethrough to bias the seal assembly 500 to the left and the right, in accordance with the present disclosure.

In FIG. 6, the surgical instrument 610 is moved to the right, thus biasing the upper housing member 410, the lower housing member 420, and the instrument seal in a first direction, “x.” When shifted to the right, the receiving member 430 shifts accordingly and gap 615 is maximized. In FIG. 7, the surgical instrument 610 is moved to the left, thus biasing the upper housing member 410, the lower housing member 420, and the instrument seal in a second direction, “y,” the second direction being generally opposite to the first direction, “x.” When shifted to the left, the receiving member 430 shifts accordingly and gap 715 is maximized. Thus, when the at least one surgical object 610 is inserted through the receiving member 430, the upper housing member 410, the lower housing member 420, and the first seal 415 are displaced in unison with respect to the central longitudinal axis “b” (see FIG. 4). The upper housing member 410, the lower housing member 420, and the first seal 415 may be generally horizontally displaced within the seal assembly 500. However, it is also contemplated that the upper housing member 410, the lower housing member 420, and the first seal 415 may be rotationally displaced (in a swinging motion) within the seal assembly 500. When displaced, it is contemplated that the lower housing member 420 does not contact the inner surface 475 of the housing 440.

In operation or in use, referring to FIGS. 4 to 7, when a surgical instrument 610 is inserted through the receiving member 430, the surgical instrument only engages the first seal 415. The second seal 425 is positioned on the exterior of the receiving member 430 in the gap 435. As the surgical instrument 610 is moved to the left and to the right, the upper housing member 410 shifts or is displaced to the left or to the right in conjunction with the lower housing member 420 and the first seal 415. In other words, the surgical instrument 610 biases the upper housing member 410 to move in relation to the receiving member 430. However, insufflation gases are prevented from escaping because of the positioning of the second seal 425 directly between the upper housing member 410 and the receiving member 430. In particular, the second seal 425 rests on a ledge 490 within the gap 435. The upper component or protrusion 412 slidably engages the second seal 425 as the surgical instrument 610 moves in generally horizontal directions, shown as directions “x” and “y” in FIG. 4. The second seal 425 always contacts or engages the ledge 490. Thus, the second seal 425 moves within a confined space defined by the gap 435. Additionally, the lower section 433 of the receiving member 430 moves within a confined space defined by the vertical portions of the upper housing member 410. It is contemplated that the receiving member 430 moves in a horizontal direction and is not displaced up or down (i.e., vertically) along the longitudinal axis “b,” see FIG. 4. The relative movement of the first seal 415 to the second seal 425 prevents insufflation gasses from escaping from space 485 (see FIG. 4). The first seal 415 is separated from the second seal 425 by the lower section 433 of the receiving member 430. The first seal 415 and the second seal 425 move within the confines of the housing 440 in a horizontal or rotational manner (e.g., swiveling or swinging from left to right) as the surgical instrument 610 is manipulated by a user (e.g., a surgeon). Once the surgical instrument 610 is removed from the receiving member 430, all the components within the assembly 400 acquire or revert to their initial unbiased positions (see FIG. 4).

Therefore, in summary, in some embodiments in accordance with the present disclosure a floating or rotating instrument seal assembly is presented that is biased by the instrument seal or the first seal so as to provide a sealing relationship with an inner surface of a housing of a seal assembly connected to a surgical access device having a cannula assembly. Additionally, the second seal (e.g., an O-ring) provides a sealing interface to a top portion of the seal assembly. The first seal and the second seal (e.g., an O-ring) are permitted to float in a generally horizontal motion. Stated differently, the first seal and the second seal are suspended within the confines of an upper housing member, a lower housing member, and a receiving member, such that the upper housing member, the lower housing member, and the first seal move in unison within the seal assembly when a surgical instrument is inserted therethrough. The first seal provides a biasing force to maintain the second seal in contact with an upper portion (or ceiling) of the upper housing member. Moreover, no interface is provided between the first seal and the duckbill seal.

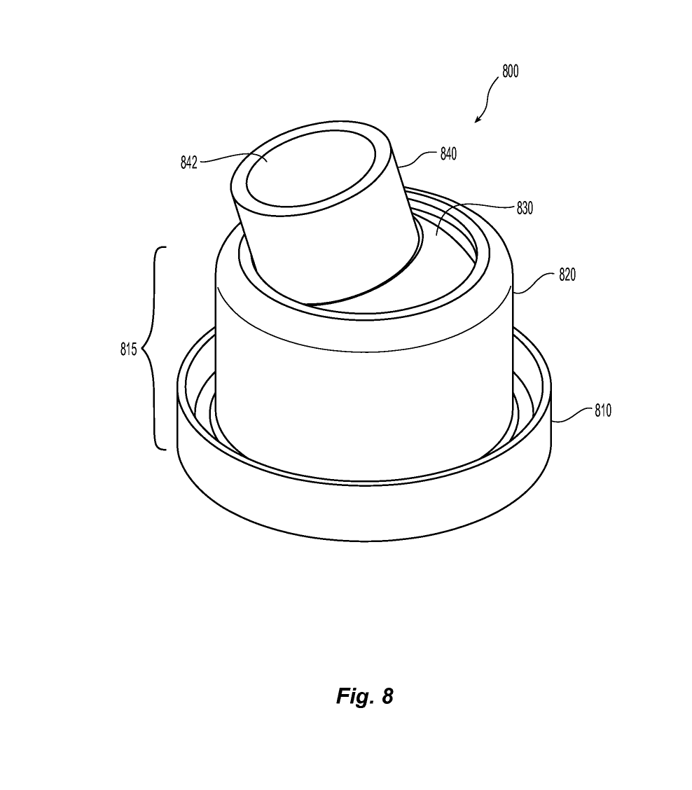

Referring to FIG. 8, a perspective view of a spherical member 830 positioned within a housing 815, in accordance with a second embodiment of the present disclosure is presented.

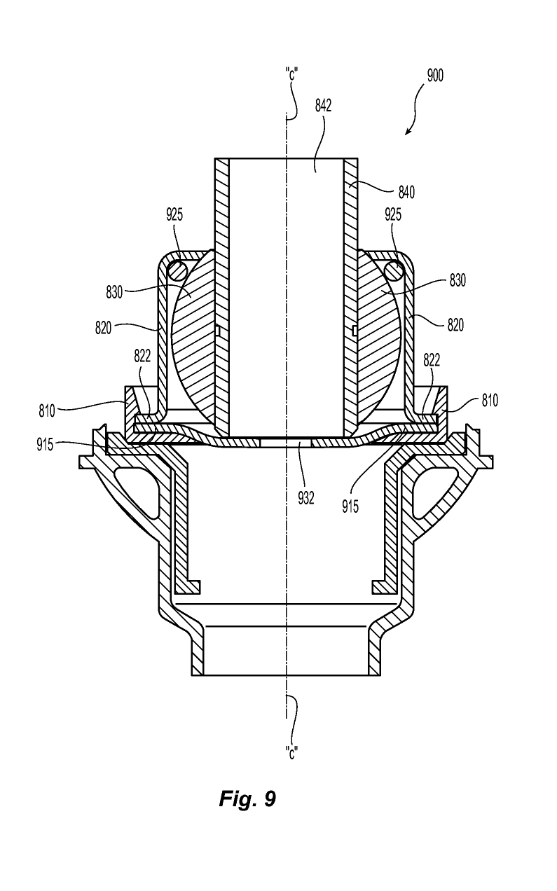

Referring to FIG. 8, a seal assembly 800 includes a spherical member 830 positioned within a housing 815 formed of an upper housing member 820 and a lower housing member 810, the spherical member 830 having a longitudinal passage 842 for receiving at least one surgical object therethrough. The longitudinal passage 842 may be a lumen 840 allowing access through opening 932 of the first seal 915 (see FIGS. 9 to 11).

Referring to FIG. 9, a cross-sectional view of a seal assembly 900 incorporating the spherical member 830 of FIG. 8, in accordance with the present disclosure is presented. The spherical member 830 defines a central longitudinal axis “c.”

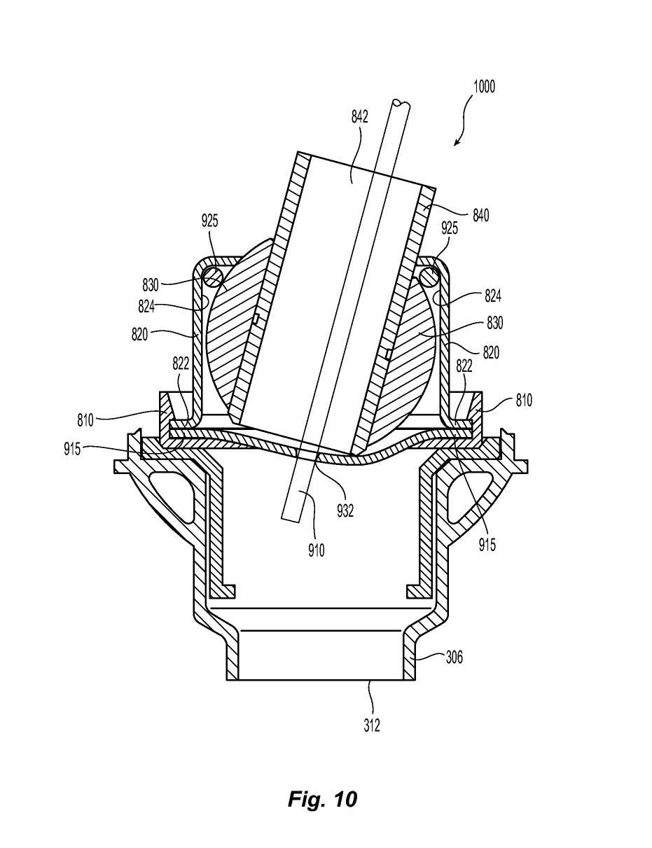

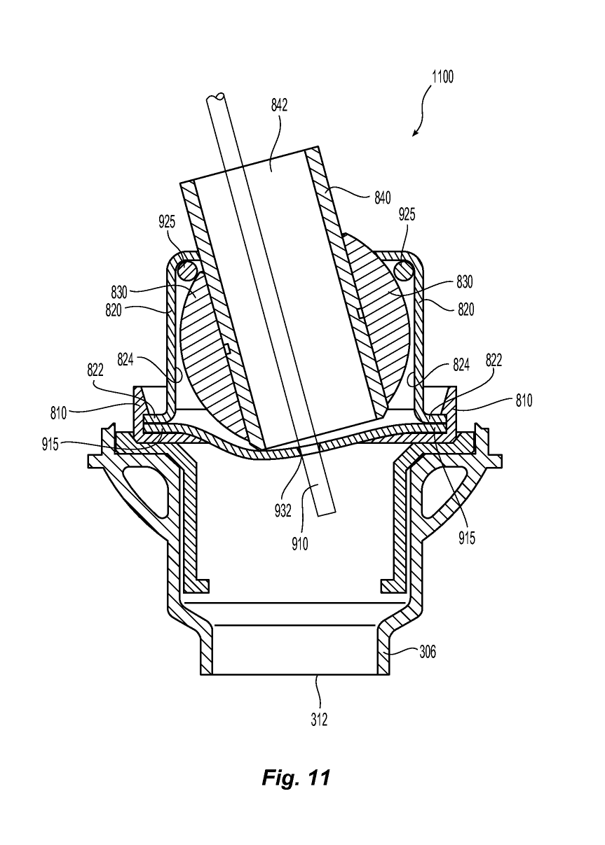

Referring to FIGS. 10 and 11, are side cross-sectional views 1000, 1100 of the seal assembly 900 of FIG. 9 connected to a cannula assembly, where a surgical instrument 910 is inserted therethrough to bias the spherical member 830 to the left and the right, in accordance with the present disclosure.

With reference to FIGS. 9 to 11, a first seal 915 is disposed within the seal assembly 900, the first seal 915 secured between the upper housing member 820 and the lower housing member 810 and a second seal 925 disposed within the seal assembly 900, the second seal 925 secured between the upper housing member 820 and the spherical member 830. The first seal 915 provides a biasing force to move the spherical member 830 toward second seal 925 to provide a sealing relationship therebetween. The first seal 915 is a substantially flat instrument seal, whereas the second seal 925 is an O-ring. A distal end 822 of the upper housing member 820 contacts a portion of the first seal 915. The distal end 822 comes in contact with the lower housing member 810 to provide a secure connection between the upper housing member 820 and the lower housing member 810.

Once assembled, the upper housing member 820, the lower housing member 810, and the first seal 915 function as a single unitary component. In contrast, the spherical member 830 rotates independently of the upper housing member 820, the lower housing member 810, and the first seal 915. The second seal 925 slidingly engages an outer surface of the spherical member 830 when the spherical member 830 swivels within the upper housing member 820 of the seal assembly 900, such that the second seal 925 prevents escape of insufflation gases.

In FIG. 10, a distal end of the surgical instrument 910 is shown biased to the left by a user after it is inserted through the passage 842 and through opening 932 located at the first seal 915. In FIG. 11, a distal end of the surgical instrument 910 is shown biased to the right by a user after it is inserted through the passage 842 and through opening 932 located in the first seal 915. As the surgical instrument 910 moves within the passage 842 extending through the spherical member 830, the second seal 925 provides a secure sealing relationship between the upper housing member 820 and the spherical member 830. The first seal 915 provides a biasing force to maintain the second seal 925, via the spherical member 830, against the top portion of the upper housing member 820 to provide a secure sealing relationship therebetween.

Moreover, the spherical member 830 is adapted and dimensioned to encompass the entire interior portion of the upper housing member 820. However, it is contemplated that the spherical member 830 does not contact the interior walls 824 of the upper housing member 820 in order to reduce the frictional relationship therebetween. The spherical member 830 may be constructed, for example, from a rigid plastic material, having a lubricating coating to facilitate rotational motion.

Therefore, in summary, in accordance with some embodiments of the present disclosure a rotating instrument seal assembly is presented that is biased by the instrument seal or the first seal so as to provide a sealing relationship with an inner surface of a housing of a seal assembly connected to a surgical access device having a cannula assembly. Additionally, the second seal (e.g., an O-ring) provides a sealing interface to a top portion of the seal assembly. The first seal provides a biasing force to maintain the second seal in sealing contact with an upper portion (or ceiling) of the upper housing member. Moreover, no interface is provided between the first seal and the duckbill seal.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of presently disclosed embodiments. Thus the scope of the embodiments should be determined by the appended claims and their legal equivalents, rather than by the examples given.

Persons skilled in the art will understand that the devices and methods specifically described herein and illustrated in the accompanying drawings are non-limiting exemplary embodiments. The features illustrated or described in connection with one exemplary embodiment may be combined with the features of other embodiments. Such modifications and variations are intended to be included within the scope of the present disclosure. As well, one skilled in the art will appreciate further features and advantages of the present disclosure based on the above-described embodiments. Accordingly, the present disclosure is not to be limited by what has been particularly shown and described, except as indicated by the appended claims.

What is claimed is:

1. A surgical access device, comprising:
a seal assembly having an upper housing member, a lower housing member, and a receiving member, the receiving member defining a central longitudinal axis and having a longitudinal passage for receiving at least one surgical object therethrough;
a first seal disposed within the seal assembly, the first seal secured between the upper housing member and the lower housing member; and
a second seal disposed within the seal assembly, the second seal movably positioned between the upper housing member and the receiving member;
wherein the first seal provides a biasing force to maintain the second seal in contact with the upper housing member to provide a sealing relationship therewith.

2. The surgical access device according to claim 1, wherein the first seal is a flat seal and the second seal is an O-ring.

3. The surgical access device according to claim 1, wherein the upper housing member, the lower housing member, and the first seal move in unison upon application of a lateral force.

4. The surgical access device according to claim 1, wherein the receiving member and the second seal move independently of the upper housing member, the lower housing member, and the first seal upon application of a lateral force.

5. The surgical access device according to claim 1, wherein an upper portion of the receiving member defines an angular opening therethrough to facilitate angular reception of at least one surgical object.

6. The surgical access device according to claim 1, wherein an upper portion of the receiving member is configured to be attached to a proximal end of the seal assembly.

7. The surgical access device according to claim 1, wherein a distal end of the seal assembly includes a duckbill seal.

8. The surgical access device according to claim 7, wherein a space between the first seal and the duckbill seal is devoid of any intermediate components.

9. The surgical access device according to claim 1, wherein movement of the second seal is restricted within a region defined by an upper portion of the upper housing member and a ridge of the receiving member.

10. A surgical access device, comprising:
a seal assembly including a housing having an upper housing member and a lower housing member;
a spherical member positioned within the housing, the spherical member defining a central longitudinal axis and having a longitudinal passage for receiving at least one surgical object therethrough;
a first seal disposed within the seal assembly, the first seal secured between the upper housing member and the lower housing member; and
a second seal disposed within the seal assembly, the second seal positioned between the upper housing member and the spherical member;
wherein the first seal provides a biasing force to maintain the spherical member in contact with the second seal to provide a sealing relationship therebetween.

11. The surgical access device according to claim 10, wherein the first seal is a flat seal and the second seal is an O-ring.

12. The surgical access device according to claim 10, wherein the spherical member moves independently of the upper housing member, the lower housing member, and the first seal.

13. The surgical access device according to claim 10, wherein a distal end of the seal assembly includes a duckbill seal.

14. The surgical access device according to claim 13, wherein a space between the first seal and the duckbill seal is devoid of any intermediate components.

15. The surgical access device according to claim 10, wherein the second seal slidingly engages an outer surface of the spherical member when the spherical member swivels within the housing of the seal assembly.

16. An access assembly, comprising:
a housing formed of an upper retaining portion and a lower retaining portion;
a first seal secured at the lower retaining portion of the housing; and
a second seal secured at the upper retaining portion of the housing;
wherein the first seal imparts a biasing force to provide a sealing relationship between the upper retaining portion and the second seal.
